(19) United States
(12) Patent Application Publication
(10) Pub. No.: US 2010/0178369 A1
(43) Pub. Date: Jul. 15, 2010

(54) ANTIOXIDANT-STABILIZED CONCENTRATED FISH OIL

(76) Inventors: Nicole Lee Arledge, Independence, KY (US); Phillip Richard Green, Wyoming, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/354,508

(22) Filed: Jan. 15, 2009

(51) Int. Cl. A61K 36/87

(52) U.S. Cl. 424/766

(57) ABSTRACT

The present disclosure relates to antioxidant-stabilized composition containing at least one omega 3 polyunsaturated fatty acid (PUFA). The omega 3 PUFA may include eicosapentanoic acid, docosahexaneoic acid and combinations thereof. The compositions additionally contain an antioxidant package made from a mixture of: at least one form of vitamin E; ascorbyl palmitate; rosemary extract; and grape seed oil. The present disclosure further relates to a method of increasing the oxidative stability of concentrated fish oil.

### FIELD OF THE INVENTION

The present invention relates to antioxidant-stabilized compositions comprising omega 3 polyunsaturated fatty adds and methods of increasing the oxidative stability thereof.

### BACKGROUND OF THE INVENTION

Consumption of foods rich in omega 3 polyunsaturated fatty adds (PUFAs) has been associated with a variety of health benefits. Indeed, a growing body of literature suggests that intake of omega 3 PUFAs including α-linolenic acid (ALA), eicosapentaenoic acid (EPA) and docosahexaenoic acid (DHA), may afford some degree of protection against coronary heart disease.

Fish, particularly oily fish, are a good dietary source of the aforementioned omega 3 PUFAs, particularly EPA and DHA. Yet fish may also serve as a dietary source of toxins including heavy metals and fat-soluble pollutants (e.g., PCBs and dioxins) which can accumulate in the animals' skin and fat. One means of addressing this drawback has been to extract the oil from the tissues of fish and to utilize the oil itself as a dietary supplement. The extracted oil can then be processed to remove contaminants through various means, for example via molecular distillation. The extracted oil can also be processed to increase the EPA and DHA content, to produce so-called “concentrated fish oil”.

Concentrated fish oil may be consumed “as is”. Alternatively, it may be sold to the consumer in any suitable product form. Non-limiting examples of suitable product forms include encapsulation in a gelatin capsule or addition of the concentrated fish oil directly into foods. Typically, these types of products are manufactured at plants in various geographical locations, prepackaged and then distributed to various retail outlets. Consequently, the shelf-life of the products may be an issue. For example, omega 3 PUFAs may be subject to oxidation over time. This oxidation may form hydroperoxides, which can in turn decompose to produce volatile compounds having objectionable odor and flavors. Moreover, oxidation of omega 3 PUFAs may reduce their nutritional value.

A variety of antioxidants and antioxidant packages are utilized to increase the stability of omega 3 PUFAs and in turn prolong the shelf-life of the products containing them. Many of these antioxidants are synthetic. Use of synthetic compounds may have several drawbacks. For example, health conscious consumers often view consumption of synthetic compounds as undesirable. Moreover, some synthetic compounds, such as butylated hydroxytoluene (BHT) and butylated hydroxyanisole (BHA), are suspected to be carcinogenic and/or toxic. On the other hand, many of the antioxidants utilized are natural; however use of these too may have associated problems which may limit their usefulness. Non-limiting examples of such problems include imparting unwanted flavors, colors and odors to the products, particularly to the food products which contain them.

Accordingly, one object of the present invention is to provide a composition comprising concentrated fish oil and an antioxidant package that will retard the oxidation of the omega 3 PUFAs contained therein. Further, the antioxidant package may have improved antioxidant activity over other antioxidant packages so as to provide better prevention of oxidation and any associated food deterioration. This improved antioxidant activity may occur with minimization of unwanted byproducts including, but not limited to, the addition of undesirable flavors, colors and odors to the concentrated fish oil itself, or to the products in which the concentrated fish oil is present. A further object of the present invention is to utilize natural antioxidants which may inherently impart additional nutritive value to the concentrated fish oil.

### SUMMARY OF THE INVENTION

The present invention addresses the aforementioned problems by providing an antioxidant-stabilized composition containing at least one omega 3 PUFA. The omega 3 PUFA may include eicosapentanoic acid, docosahexaneoic acid and combinations thereof. The compositions further comprise: at least one form of vitamin E; ascorbyl palmitate; rosemary extract; and grape seed oil. The omega 3 PUFAs may be derived from fish oil, particularly concentrated fish oil. The compositions may additionally comprise one or more masking agents. A non-limiting example of a useful masking agent is vanillin.

In some embodiments, the anti-oxidant stabilized compositions may comprise the following by weight percentage of the composition: (a) from about 20% to about 33% eicosapentanoic acid; (b) from about 11% to about 30% docosahexaneoic acid; (c) from about 0.1% to about 0.2% mixed tocopherols; (d) from about 0.02% to about 0.025% ascorbyl palmitate; (e) from about 0.05% to about 0.1% rosemary extract; and (f) from about 10% to about 20% grape seed oil.

The present invention further addresses the aforementioned problems by providing a method of increasing the Oxidative Stability Index (OSI) of concentrated fish oil. The method comprises the step of adding an antioxidant package to the concentrated fish oil. The antioxidant package comprises: at least one form of vitamin E; ascorbyl palmitate; rosemary extract; and grape seed oil.

### DETAILED DESCRIPTION OF THE INVENTION

“Comprising” as used herein means that various components, ingredients or steps can that be conjointly employed in practicing the present invention. Accordingly, the term “comprising” encompasses the more restrictive terms “consisting essentially of” and “consisting of”. The present compositions can comprise, consist essentially of, or consist of any of the required and optional elements disclosed herein.

“Fish Oil” as used herein is meant to encompass oil derived from fish and/or marine organism(s) in general including, but not limited to, algae.

Markush language as used herein encompasses combinations of the individual Markush group members, unless otherwise indicated.

All percentages, ratios and proportions used herein are by weight percent of the composition, unless otherwise specified. All average values are calculated “by weight” of the composition or components thereof, unless otherwise expressly indicated.

All numerical ranges disclosed herein, are meant to encompass each individual number within the range and to encompass any combination of the disclosed upper and lower limits of the ranges.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

### I. COMPOSITION

One aspect of the present invention includes compositions comprising: A. concentrated fish oil; B. at least one omega 3 PUFA; C. at least one form of Vitamin E; D. ascorbyl palmitate; E. rosemary extract; F. grape seed oil; G. masking agent; and optionally, H. water. Some compositions according to the present invention may contain no added phospholipid.

Each of “A” through “H” is discussed in further detail below.

A. Concentrated Fish Oil

Fish oil may be harvested by one of skill in the art from any suitable source. Non-limiting examples of omega 3 PUFA rich sources include: abalone scallops, albacore tuna, anchovies, catfish, clams, cod, fem fish, herring, lake trout, mackerel, menhaden, orange roughy, salmon, sardines, sea mullet, sea perch, shark, shrimp, squid, trout and tuna. The harvested fish oil may then be concentrated to increase its omega 3 PUFA content using any suitable method known to one of skill in the art. As used herein, “concentrated fish oil” refers to fish oil comprising the following (by weight percentage of the fish oil): from about 20% to about 70% EPA and/or from about 11% to about 70% DHA.

Compositions according to the present invention may comprise concentrated fish oil by weight percentage of the composition: from about 70% to about 95%, from about 75% to about 92.5%, or from about 79% to about 90%.

B. Omega 3 PUFAs

“Omega 3 PUFAs”, which are also popularly referred to as “3 fatty acids” or “ω-3 fatty acids” are a family of PUFAs that have in common a final carbon-carbon double bond in the n-3 position; that is, the third bond from the methyl end of the fatty acid. Omega 3 PUFAs include, but are not limited to: ALA, EPA and DHA, all of which are polyunsaturated.

Compositions according to the present invention may comprise by weight percentage of the composition: from about 20% to about 70%, from about 20% to about 50%, or from about 20% to about 33% of EPA; and/or from about 11% to about 70%, from about 11% to about 50%, or from about 11% to about 30% of DHA.

C. Vitamin E

Vitamin E, a natural compound, exists in eight different forms, including four tocopherols and four tocotrienols. All feature a chromanol ring, with a hydroxyl group that can donate a hydrogen atom to reduce free radicals and a hydrophobic side chain which allows for penetration into biological membranes. Both the tocopherols and tocotrienols occur in alpha (α), beta (β), gamma (γ) and delta (δ) forms, which is determined by the number of methyl groups on the chromanol ring. Each form, which has slightly different biological activity, has the following general formula (I), as shown below:

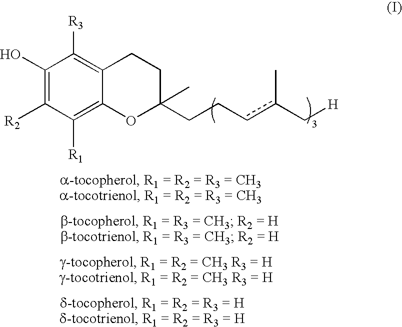

Thus “mixed tocopherols” refers to any combination of more than one of the forms of tocopherol, “mixed tocotrienols” refers to any combination of more than one of the forms of tocopherols and “mixed vitamin E” refers to any combination of more than one of the forms of vitamin E.

Compositions according to the present invention may comprise by weight percentage of the composition: from about 0.025% to about 0.4%, from about 0.075% to about 0.3%, or from about 0. 1% to about 0.2% of at least one form of vitamin E.

D. Ascorbyl Palmitate

Ascorbyl palmitate is an ester formed from ascorbic acid and palmitic add creating a fat-soluble form of vitamin C. In addition to its use as a natural source of vitamin C, it may be used as an antioxidant food additive. Without wishing to be bound by theory, it is believed that ascorbyl palmitate may act as an oxygen scavenger and may also act to regenerate phenolic antioxidants including, but not limited to, the different forms of vitamin E.

The solubility of ascorbyl palmitate in fish oil may be limited. Consequently, consideration of solubility may dictate the ranges of useful weight percentages of ascorbyl palmitate in the present compositions. Compositions according to the present invention may comprise by weight percentage of the composition from about 0.0075% to about 0.025% or from about 0.02% to about 0.025% of ascorbyl palmitate.

E. Rosemary Extract

Rosemary extract is a natural and known material extracted from the rosemary plant. The term “rosemary extract” as used herein is a generic term describing a number of different chemical compositions that may contain several different active components. Among the common components that are found in rosemary extract are: caffeic acid; carnosol; carnosic acid; methoxy carnosic acid; rosmarinic acid; rosmanol; and rosmaridiphenol; all of which may be present in different proportions depending on the individual extract. Numerous rosemary extracts are available commercially, and any one or more can be used in the present invention.

Rosemary extract is believed to be a potent anti-oxidant containing anti-inflammatory agents. Rosemary is commonly used as a flavor enhancer.

Compositions according to the present invention may comprise by weight percentage of the composition: from about 0.025% to about 0.2%, from about 0.04% to about 0.075% or from about 0.05% to about 0.1% of rosemary extract.

F. Grape seed Oil

Grape seed oil (also called grapeseed oil or grape oil) may be derived from vegetable oil that is pressed from the seeds of one or more varieties of Vitis vinifera grapes. Grape seed oil may contain a variety of PUFAs, which may vary according to carbonic chain length and/or degree of saturation. Non-limiting examples of PUFAs which are found in grape seed oil, as well as the ranges of weight percentage in which they may be present in the grape seed oil are found in TABLE I:

TABLE I

| | | Average Weight Range |
|---|---|---|
| | Carbon Chain | Percentages of Grape Seed |
| | Length:Number | Oil |
| PUFA Name | of Double Bonds | (wt %) |
| Lauric | 12:0 | From 0 to about 4; from 0 to |
| | | about 5; or from 0 to about 6 |
| Myristic | 14:0 | From 0 to about 1.5; from 0 to |
| | | about 1.0; or from 0 to about |
| | | 2.0 |
| Palmitic | 16:0 | From about 5.0 to about 9.0; |
| | | from about 5.5 to about 8.5; or |
| | | from 6.0 to about 8.0 |
| Stearic | 18:0 | From about 2.0 to about 6.0; |
| | | from about 2.5 to about 5.5; or |
| | | from about 3.0 to about 4.2 |
| Palmitoleic | 16:1 | From 0 to about 1.0; from 0 to |
| | | about 0.5; or from 0 to about |
| | | 0.1 |
| Oleic | 18:1 | From about 12.0 to about 26.0; |
| | | from about 13.0 to about 25.0; |
| | | or from about 14.9 to about |
| | | 24.6 |
| Linoleic | 18:2 | From about 60.0 to about 80.0; |
| | | from about 65.0 to about 75.0; |
| | | or from about 70.0 to about |
| | | 73.3 |
| Linolenic | 18:3 | From 0 to about 1.0; from 0 to |
| | | about .75; or from 0 to about |
| | | 0.5 |

Numerous grape seed oils are available commercially, and any one or more can be used in the present invention.

It is believed that grape seed oil not only acts as an antioxidant in the compositions of the present invention, but it may also impart further nutritive value thereto. For example, the phenolics which may be present in grape seed oil are believed to inhibit human low-density lipoprotein (LDL) oxidation in vitro. Moreover, the monomeric phenolic compounds which may be present in fish oil including, but not limited to: (+)-catechins; (−)-epicatechin; and (−)-epicatechin-3-O-gallate; dimeric, trimeric and tetrameric proanthocyanidins; may act as antimutagenic and antiviral agents. Indeed, recognition of such health benefits of catechins and proanthocyanidins has led to the use of grape seed extract as a dietary supplement.

Compositions according to the present invention may comprise by weight percentage of the composition: from about 5% to about 30%, from about 7% to about 25% or from about 10% to about 20% of grape seed oil.

G. Masking Agent

“Masking agent” as used herein refers to a natural compound or combination of compounds which improves the odor and flavor of the compositions of the present invention, without compromising their stability. Some masking agents may also provide antioxidant activity.

Any suitable masking agent may be utilized by one of skill in the art. Non-limiting examples of suitable masking agents include: vanillin; hazel nut oil; palm oil; pecan oil; sunflower oil; pumpkin seed oil; aloe vera; avocado pear oil; evening primrose oil; kukui nut oil; peanut oil; pistachio nut oil; rosehip oil; sesame oil; walnut oil; natural cocoa butter; jojoba oil; almond sweet oil; flax seed oil; turmeric essential oil; wintergreen essential oil; tangerine essential oil; thyme sweet essential oil; tea tree essential oil; spearmint essential oil; onion essential oil; oregano essential oil; marjoram sweet essential oil; vanilla essential oil; tarragon essential oil; thyme white essential oil; orange sweet essential oil; lime essential oil; basil essential oil; almond butter essential oil; anise seed essential oil; essential oil; celery seed essential oil; cinnamon essential oil; caraway essential oil; cinnamon leaf essential oil; cumin seed essential oil; grapefruit white; lemon balm essential oil; and combinations thereof.

One of skill in the art may choose the weight percentage of masking agent to be utilized in the present compositions depending upon the final characteristics that are desired. Compositions according to the present invention may comprise by weight percentage of the composition: from about 0.01% to about 1%, from about 0.02% to about 0.1% or from about 0.025% to about 0.05% of masking agent.

H. Water

Water may not be added to the compositions of the present invention. Water may inherently be present in one of the components of the compositions. Compositions of the present invention may contain 0 to about 0.1% by weight percentage of the composition.

### II. METHODS

A. Making the Composition

The compositions of the present invention may be made using any suitable means by one of skill in the art. One useful means is described as follows. At least one form of vitamin E, ascorbyl palmitate and rosemary extract are added into grape seed oil to form a pre-mix. The pre-mix is then added to the fish oil and mixed together, for example in a circulated tank.

B. Measuring the Oxidative Stability Index (OSI)

Oxidation stability is measured using the Omnion Oxidative Stability Instrument (OSI-24) from Omnion, Inc. (Rockland, Mass.) per the User's Manual (p. 13-14).

The Oxidation Stability Index (OSI) is evaluated using the Oxidation Stability Index (OSI) method, a method approved by the American Oil Chemists' Society (AOCS Official Method Cd 12b-92). This method measures the period of time during which oils are resistant to oxidation. After this period of time, or the induction period, the rate of oxidation accelerates rapidly. During the OSI procedure, a stream of air is passed through an oil sample, which is heated to 65° C., and the effluent air from the oil sample is bubbled through a test vessel containing deionized water, the conductivity of which is continuously monitored over time. As the oil oxidizes, volatile organic compounds are generated and become trapped in the water, thereby increasing its conductivity. The OSI value is defined as the induction period in hours and mathematically represents the inflection point (second derivative) of the conductivity curve that reflects the maximum change in the oxidation rate. The higher the OSI value, the more stable the oil.

### III. EXAMPLES

The invention will now be described with reference to the following examples. The OSI of each sample composition found below is measured and evaluated as described above. Note that the OSI sample composition size is 5+/−0.2 grams.

The OSI values for comparative examples 1-5 are found in TABLE II.

TABLE II

| EXAMPLE | SAMPLE COMPOSITION (weight | OSI (hours |
|---|---|---|
| NUMBER | percentage of the sample composition) | @ 65° C.) |
| 1 | 100% Concentrated Fish Oil¹ | 5.65 |
| 2 | 90% Concentrated Fish Oil¹, 10% Rice | 10.25 |
| | Bran Oil² | |
| 3 | 90% Concentrated Fish Oil¹, 10% Soybean | 10.78 |
| | Oil³ | |
| 4 | 90% Concentrated Fish Oil¹, 10% Grape | 10.58 |
| | Seed Oil⁴ | |
| 5 | 80% Concentrated Fish Oil¹, 20% Grape | 12.5 |
| | Seed Oil⁴ | |

¹Omegaphyl 3020 (Twin Rivers Technology Natural Ingredients (TRT-NI), Cincinnati, OH); contains 30 weight % EPA and 20 weight % DHA
²RBDD Rice Bran Oil (Rito, Inc., San Franscisco, CA)
³Soybean Oil (Smuckers/Crisco, Orrville, OH)
⁴Delicate Grape Seed Oil (La Tourangelle, Woodland, CA)

The following is apparent from TABLE II. A composition comprising only concentrated fish oil that is not treated with antioxidants has an OSI of 5.65 (example 1). A composition comprising concentrated fish oil that is treated with of a variety of oils known to have antioxidant properties, raises the OSI of the resulting composition from about 10 to about 12.5 OSI (examples 2-4). By doubling the amount of grape seed oil in the composition to 20%, the OSI of the resulting composition is increased to about 15% (example 5).

The OSI values for comparative examples 6-8 are found in TABLE III.

TABLE III

| EXAMPLE | SAMPLE COMPOSITION (weight | OSI (hours |
|---|---|---|
| NUMBER | percentage of the sample composition) | @ 65° C.) |
| 6 | 99.8% Concentrated Fish Oil¹, 0.2% Mixed | 15.15 |
| | Tocopherols⁵ | |
| 7 | 99.975% Concentrated Fish Oil¹, 0.025% | 9.28 |
| | Ascorbyl Palmitate⁶ | |
| 8 | 99.9% Concentrated Fish Oil¹, 0.1% | 14 |
| | Rosemary Extract⁷ | |

⁵Decanox Mixed Tocopherols (ADM, Erith, UK)
⁶6-O-Palmitoyl-L-ascorbic acid (Sigma Aldrich, St. Louis, MO)
⁷Dadex RE-O (Nealanders International, Inc., Oakville, ON)

The following is apparent from TABLE III. A composition comprising concentrated fish oil and 0.2% mixed tocopherols has an OSI of 15.15, as compared to the OSI of 5.65 for unstabilized concentrated fish oil (examples 6 and 1, respectively). A composition comprising concentrated fish oil and 0.025% ascorbyl palmitate has an OSI of 9.28 (example 7). A composition comprising concentrated fish oil and 0. 1% Rosemary Extract has an OSI of 14.

The OSI for comparative examples 1, 6 and 9-12 are found in TABLE IV.

TABLE IV

| | | OSI |
|---|---|---|
| EXAMPLE | SAMPLE COMPOSITION | (hours @ |
| NUMBER | (weight percentage of the sample composition) | 65° C.) |
| 12 | 79.675% Concentrated Fish Oil¹, 0.2% Mixed | 53 |
| | Tocopherols⁵, 0.025% Ascorbyl Palmitate⁶, | |
| | 0.1% Rosemary Extract⁷, 20% Grape Seed Oil | |
| 11 | 99.675% Concentrated Fish Oil¹, 0.2% Mixed | 31 |
| | Tocopherols⁵, 0.025% Ascorbyl Palmitate⁶, | |
| | 0.1% Rosemary Extract⁷ | |
| 10 | 99.725% Concentrated Fish Oil¹, 0.2% Mixed | 25 |
| | Tocopherols⁵, 0.025% Ascorbyl Palmitate⁶, | |
| | 0.05% Rosemary Extract⁷ | |
| 9 | 99.775% Concentrated Fish Oil¹, 0.2% Mixed | 19 |
| | Tocopherols⁵, 0.025% Ascorbyl Palmitate⁶ | |
| 6 | 99.8% Concentrated Fish Oil¹, 0.2% Mixed | 15 |
| | Tocopherols⁵ | |
| 1 | 100% Concentrated Fish Oil¹ | 5.5 |

It is apparent from TABLE IV, that the composition of example 12, which contains concentrated fish oil as well as a combination of: 0.2% mixed tocopherols; 0.025% ascorbyl palmitate; 0.1% rosemary extract; and 20% grape seed oil, provides for an antioxidant-stabilized concentrated fish oil having a surprisingly high OSI of 53. It is also apparent that the OSI of concentrated fish oil is increased by approximately 10× (i.e., 10-fold).

TABLE V contains examples 13-20, which are different embodiments of the compositions of the present invention.

TABLE V

| | Composition Weight % | | | | | | | |
|---|---|---|---|---|---|---|---|---|
| Concentrated Fish | Example | Example | Example | Example | Example | Example | Example | Example |
| Oil | 13 | 14 | 15 | 16 | 17 | 18 | 19 | 20 |
| EPA | 20 | 30 | 20 | 30 | 26 | 33 | 26 | 33 |
| DHA | 30 | 20 | 30 | 20 | 20 | 11 | 20 | 11 |
| Vitamin E | 0.1 | 0.2 | 0.2 | 0.1 | 0.1 | 0.2 | 0.2 | 0.1 |
| Ascorbyl Palmitate | 0.02 | 0.025 | 0.025 | 0.02 | 0.02 | 0.025 | 0.025 | 0.02 |
| Rosemary Extract | 0.05 | 0.1 | 0.1 | 0.05 | 0.05 | 0.1 | 0.1 | 0.05 |
| Grape Seed Oil | 10 | 20 | 20 | 10 | 10 | 20 | 20 | 10 |
| Vanillin | 0.025 | 0.05 | 0 | 0 | 0.025 | 0.05 | 0 | 0.025 |
| Wintergreen | 0 | 0 | 0.05 | 0.025 | 0 | 0 | 0.05 | 0 |
| Essential Oil | | | | | | | | |
| Water | 0 | 0.1 | 0.1 | 0 | 0 | 0.1 | 0.1 | 0 |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A composition comprising at least one omega 3 polyunsaturated fatty acid selected from the group consisting of eicosapentanoic acid, docosahexaneoic acid and combinations thereof, and further comprising by weight percentage of the composition:
(a) from about 0.025% to about 0.04% of at least one form of vitamin E;
(b) from about 0.0075% to about 0.025% of ascorbyl palmitate;
(c) from about 0.025% to about 0.2% of rosemary extract; and
(d) from about 5% to about 30% of grape seed oil,
wherein when the composition comprises eicosapentanoic acid, the composition comprises by weight percentage from about 20% to about 70% of eicosapentanoic acid and when the composition comprises docosahexaneoic acid, the composition comprises by weight percentage from about 11% to about 70% of docosahexaneoic acid.

2. The composition of claim 1, said composition comprises eicosapentanoic acid.

3. The composition of claim 1, said composition comprises docosahexaneoic acid.

4. The composition of claim 1, further comprising a masking agent.

5. The composition of claim 4, wherein said masking agent is vanillin.

6. The composition of claim 1, wherein said at least one form of vitamin E is selected from the group consisting of: α-tocopherol; β-tocopherol; γ-tocopherol; δ-tocopherol; α-tocotrienol; β-tocotrienol; γ-tocotrienol; δ-tocotrienol; and combinations thereof.

7. The composition of claim 1, having an Oxidative Stability Index of at least about 50 hours.

8. The composition of claim 1, having an Oxidative Stability Index of from about 5.5 to about 53 hours.

9. The composition according to claim 1, wherein said omega 3 polyunsaturated fatty acid is present in said composition as a component of concentrated fish oil.

10. A composition comprising by weight percentage:
(a) from about 20% to about 33% eicosapentanoic acid;
(b) from about 11% to about 30% docosahexaneoic acid;
(c) from about 0.1% to about 0.2% mixed tocopherols;
(d) from about 0.02% to about 0.025% ascorbyl palmitate;
(e) from about 0.05% to about 0.1% rosemary extract; and
(f) from about 10% to about 20% grape seed oil.

11. The composition of claim 10, further comprising by weight percentage from about 0.025% to about 0.05% vanillin.

12. The composition of claim 10, wherein said eicosapentanoic acid and docosahexaneoic acid are present in said composition as a component of concentrated fish oil.

13. A method of increasing the inherent Oxidation Stability Index of concentrated fish oil by at least about 10× to a final Oxidation Stability Index, comprising the step of adding an antioxidant package to said concentrated fish oil to form an anti-oxidant stabilized composition, said antioxidant package comprising: at least one form of vitamin E; ascorbyl palmitate; rosemary extract; and grape seed oil.

14. The method of claim 13, wherein the inherent Oxidation Stability Index is from about 5 to about 7 hours and said final Oxidation Stability Index is from about 50 to about 70 hours.

15. The method of claim 13, said concentrated fish oil comprising by weight percentage from about 11% to about 75% of at least one omega 3 polyunsaturated fatty acid.

16. The method of claim 15, wherein said at least one omega 3 polyunsaturated fatty acid is selected from the group consisting of: eicosapentanoic acid; docosahexaneoic acid; and combinations thereof.

17. The method of claim 16, said concentrated fish oil comprising by weight percentage from about 20% to about 70% of eicosapentanoic acid and from about 11% to about 70% of docosahexaneoic acid.

18. The method of claim 13, further comprising the step of adding a masking agent to said concentrated fish oil.

19. The method of claim 18, wherein said masking agent is vanillin.

20. The method of claim 13, wherein said at least one form of vitamin E is selected from the group consisting of: α-tocopherol; β-tocopherol; γ-tocopherol; wherein when the composition comprises eicosapentanoic acid, the composition comprises by weight percentage from about 20% to about 70% of eicosapentanoic acid and when the composition comprises docosahexaneoic acid, the composition comprises by weight percentage from about 11% to about 70% of docosahexaneoic acid. δ-tocopherol; α-tocotrienol; β-tocotrienol; γ-tocotrienol; δ-tocotrienol; and combinations thereof.
